(19) United States
(12) Patent Application Publication
(10) Pub. No.: US 2012/0219558 A1
(43) Pub. Date: Aug. 30, 2012

(54) ANTAGONISTS OF PCSK9

(76) Inventors: Yan Ni, Westfield, NJ (US); Ayesha Sitlani, Metuchen, NJ (US); Shilpa Pandit, Edison, NJ (US); Dale Lewis, Washington Crossing, PA (US); Xun Shen, Piscataway, NJ (US); Sharon Lobo, Hackettstown, NJ (US); Timothy McCabe, Doylestown, PA (US); Jon Condra, Doylestown, PA (US); Rose Cubbon, Fanwood, NJ (US); Fubao Wang, Drescher, PA (US)

(21) Appl. No.: 13/497,663

(22) PCT Filed: Sep. 15, 2010
(86) PCT No.: PCT/US10/48849

§ 371 Date: May 18, 2012

Related U.S. Application Data
(60) Provisional application No. 61/245,691, filed on Sep. 25, 2009.

(51) Int. Cl. A61K 39/395; C12N 15/13; C12N 1/21; C12N 15/63; C12P 21/08; C07K 16/40; A61P 7/00

(52) U.S. Cl. 424/146.1; 530/388.26; 536/23.53; 435/320.1; 435/69.6; 435/252.33

(57) ABSTRACT

Antagonists of human proprotein convertase subtilisin-kexin type 9 (PCSK9) are disclosed. Said antagonists are effective in the inhibition of PCSK9 function and thereby provide compositions of matter useful for the treatment of conditions associated with PCSK9 activity. The present invention further discloses nucleic acids encoding PCSK9 antagonists as well as methods of making and using PCSK9 antagonists.

### BACKGROUND OF THE INVENTION

Proprotein convertase subtilisin-pexin type 9 (PCSK9), also known as neural apoptosis- regulated convertase 1 (NARC-1), is a proteinase K-like subtilase which was identified as the 9^{th} member of the secretory subtilase family (Seidah, N. G., et al., 2003 PROC NATL ACAD SCI USA 100:928-933). PCSK9 is expressed in cells capable of proliferation and differentiation such as hepatocytes, kidney mesenchymal cells, intestinal ileum, colon epithelia and embryonic brain telencephalic neurons (Seidah et al., 2003). The gene for human PCSK9 has been sequenced and is about 22-kb long, with 12 exons that encode a 692 amino acid protein (NP_{—}777596.2).

PCSK9 has been implicated in cholesterol homeostasis, as it appears to have a specific role in cholesterol biosynthesis or uptake. In a study of cholesterol-fed rats, it was reported that PCSK9 was downregulated in a similar manner to other genes involved in cholesterol biosynthesis, (Maxwell et al., 2003 J. LIPID RES. 44:2109-2119), Further, the expression of PCSK9 was regulated by sterol regulatory element-binding proteins (SREBP), as seen in other genes involved in cholesterol metabolism (Maxwell, et al., 2003).

PCSK9 expression has been found to be upregulated by statins in a manner attributed to the cholesterol-lowering effects of the drugs (Dubuc et al., 2004 ARTERIOSCLER. THROMB. VASC. BIOL. 24:1454-1459). Adenoviral expression of PCSK9 results in a time-dependent increase in circulating low density lipoprotein (LDL) (Benjannet et al., 2004 J. BIOL. CHEM. 279:48865-48875), and mice with PCSK9 gene deletions have increased levels of hepatic LDL receptors (LDLR) and clear LDL from the plasma more rapidly (Rashid et al., 2005 PROC. NATL. ACAD. SCI. USA 102:5374-5379). Medium from HepG2 cells which are transiently transfected with PCSK9 is found to reduce the amount of cell surface LDLRs and internalization of LDL when transferred to untransfected HepG2 cells (Cameron et al., 2006 HUMAN MOL. GENET. 15:1551-1558). Additionally, purified PCSK9 added to the medium of HepG2 cells reduced the number of cell-surface LDLRs in a dose- and time-dependent manner (Lagace et al., 2006 J. CLIN. INVEST. 116:2995-3005),

A number of mutations in the gene PCSK9 have been associated with autosomal dominant hypercholesterolemia (ADH), an inherited metabolism disorder which is characterized by marked elevations of LDL particles in the plasma that can lead to premature cardiovascular failure (e.g., Abifadel et al., 2003 NATURE GENETICS 34:154-156; Timms et al., 2004 HUM. GENET. 114:349-353; Leren, 2004 CLIN. GENET. 65:419-422).

PCSK9 therefore appears to play a role in the regulation of LDL production. Expression or upregulation of PCSK9 is associated with increased plasma levels of LDL cholesterol, and inhibition or the lack of expression of PCSK9 is associated with low LDL cholesterol plasma levels and lower levels of LDL cholesterol associated with sequence variations in PCSK9 confer protection against coronary heart disease (Cohen, et al., 2006 N. ENGL. J. MED. 354:1264-1272). This is of significance as clinical trial data has demonstrated that reductions in LDL cholesterol levels are related to the rate of coronary events (Law et al., 2003 BMJ 326:1423-1427). Moderate lifelong reduction in plasma LDL cholesterol levels has been shown to be substantially correlated with a substantial reduction in the incidence of coronary events (Cohen et al., 2006), even in populations with a high prevalence of non-lipid-related cardiovascular risk factors. Accordingly, there is great benefit to be reaped from the managed control of LDL cholesterol levels.

It is therefore desirable to further investigate PCSK9 as a target for the treatment of cardiovascular disease. Antibodies useful as PCSK9 antagonists have been identified and have utility as therapeutic agents.

It would be further desirable to be able to identify novel PCSK9 antagonists in order to assist in the quest for compounds and/or agents effective in the treatment of cardiovascular disease. Hence, a method for measuring levels of circulating PCSK9 in a biological sample for such purposes as, e.g., assessing the effectiveness of a putative PCSK9 antagonist is of utility.

### SUMMARY OF THE INVENTION

The present invention relates to PCSK9-specific antagonists that antagonize PCSK9's inhibition of cellular LDL uptake, wherein said antagonists comprise a monoclonal antibody comprising a light chain polypeptide having the amino acid sequence of SEQ ID NO: 3 and a heavy chain polypeptide having the amino acid sequence of SEQ ID NO: 4.

The present invention relates to PCSK9-specific antagonists that antagonize PCSK9's inhibition of cellular LDL uptake, wherein said antagonists comprise a monoclonal antibody comprising a light chain polypeptide having the amino acid sequence of SEQ ID NO: 7 and a heavy chain polypeptide having the amino acid sequence of SEQ ID NO: 8.

The present invention further relates to a composition comprising a PCSK9-specific antagonist such as described supra and a pharmaceutically acceptable carrier.

The present invention also provides a method for antagonizing PCSK9 function which comprises the step of administering a PCSK9-specific antagonist to cells, tissues, or human or animal subjects.

The present invention further furnishes a use of a PCSK9-specific antagonist in the manufacture of a medicament for ameliorating a disorder, condition or disease caused and/or exacerbated by PCSK9 function.

The present invention provides isolated nucleic acids coding for the heavy and light chain polypeptides of SEQ ID NOs: 1, 2, 5 and 6.

The present invention also provides vectors comprising isolated nucleic acids coding for the heavy and light chain polypeptides of SEQ ID NOs: 1, 2, 5 and 6 as well as host cells comprising said vectors.

The present invention also furnishes a method for producing a PCSK9-specific antagonist which comprises: (a) culturing a population of cells comprising host cells comprising vectors having isolated nucleic acids coding for the heavy and light chain polypeptides of SEQ ID NOs: 3 and 4 or 5 and 6 under conditions appropriate for production of the PCSK9-specific antagonist; and

(b) isolating the PCSK9-specific antagonist produced.

### BRIEF DESCRIPTION OF THE DRAWINGS

FIG. 1 shows that the E07 Fab is a partial inhibitor of PCSK9 function.

FIG. 2 shows that E07, G08 and H23 (Fab) do not compete with 1B20 IgG for PCSK9 binding.

### DETAILED DESCRIPTION OF THE INVENTION

The present invention provides antagonists of PCSK-9 function which are monoclonal antibodies. In a preferred embodiment, there is provided an antagonist of PCSK-9 function which comprises a light chain polypeptide comprising CDR1, CDR2 and CDR3 of SEQ ID NO: 3 and a heavy chain polypeptide comprising CDR1, CDR2, and CDR3 of SEQ ID NO: 4.

The present invention further furnishes a use of a PCSK9-specific antagonist in the manufacture of a medicament for ameliorating a disorder, condition or disease caused and/or exacerbated by PCSK9 function. The utility of these disclosed antagonists is directly measurable by assays readily available to the skilled artisan. Means for measuring LDL uptake are described in the literature (see, e.g., Barak & Webb, 1981 J. Cell Biol. 90:595-604, and Stephan & Yurachek, 1993 J. Lipid Res. 34:325330). In addition, means for measuring LDL cholesterol in plasma is well described in the literature (see, e.g., McNamara et al., 2006 Clinica Chimica Acta 369:158-167).

The present invention provides isolated nucleic acids coding for the heavy and light chain polypeptides of SEQ ID NOs: 1, 2, 5 and 6.

The present invention also provides vectors comprising isolated nucleic acids coding for the heavy and light chain polypeptides of SEQ ID NOs: 1, 2, 5 and 6 as well as host cells comprising said vectors.

The present invention also furnishes a method for producing a PCSK9-specific antagonist which comprises: (a) culturing a population of cells comprising host cells comprising vectors having isolated nucleic acids coding for the heavy and light chain polypeptides of SEQ ID NOs: 3 and 4 or 7 and 8 under conditions appropriate for production of the PCSK9-specific antagonist; and

(c) isolating the PCSK9-specific antagonist produced.

In another aspect, the present invention provides a method for identifying, isolating, quantifying or antagonizing PCSK9 in a sample of interest using one or more PCSK9-specific antagonists of the present invention. The PCSK9-specific antagonists may be utilized as research tools in immunochemical assays, such as Western blots, ELISAs, radioimmunoassay, immunohistochemical assays, immunoprecipitations, or other immunochemical assays known in the art (see, e.g., Immunological Techniques Laboratory Manual, ed. Goers, J. 1993, Academic Press) or various purification protocols. The antagonists may have a label incorporated therein or affixed thereto to facilitate ready identification or measurement of the activities associated therewith. One skilled in the art is readily familiar with the various types of detectable labels (e.g., enzymes, dyes, or other suitable molecules which are either readily detectable or cause some activity/result that is readily detectable) which are or may be useful in the above protocols.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each mAb is directed against a single determinant on the antigen. In addition to their specificity, monoclonal antibodies are advantageous in that they can be synthesized by hybridoma culture, uncontaminated by other immunoglobulins. The term “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., (1975) Nature, 256:495, or may be made by recombinant DNA methods (see, e.g., U.S. Pat. No. 4,816,567 to Cabilly et al.).

Use of the term “antagonist” herein refers to the fact that the subject molecule can antagonize the functioning of PCSK9. Use of the term “antagonizing” herein refers to the act of opposing, counteracting, neutralizing or curtailing one or more functions of PCSK9. Reference herein to PCSK9 function or PCSK9 activity refers to any function or activity that is driven by, requires, or is exacerbated or enhanced by PCSK9.

As used herein, the term “isolated” describes a property as it pertains to the disclosed PCSK9-specific antagonists, nucleic acid or other that makes them different from that found in nature. The difference can be, for example, that they are of a different purity than that found in nature, or that they are of a different structure or form part of a different structure than that found in nature. A structure not found in nature, for example, includes recombinant human immunoglobulin structures including, but not limited to, recombinant human immunoglobulin structures with optimized CDRs. Other examples of structures not found in nature are PCSK9-specific antagonists or nucleic acid substantially free of other cellular material. Isolated PCSK9-specific antagonists are generally free of other protein-specific antagonists having different protein specificities (i.e., possess an affinity for other than PCSK9).

The CDR definitions arrived at and disclosed herein were defined using the Morphosys software program Sequence Analysis Software (SAS). Various other methods are available, however, to delineate and define start and end points of CDR sequences, e.g., most conspicuously, Kabat, E. A., Wu, T. T., Perry, H., Gottesman, K. and Foeller, C. (1991) Sequences of Proteins of Immunological Interest, Fifth Edition. NIH Publication No. 91-3242. While the current inventors have used the SAS software to define the CDRs, the present invention encompasses different definitions around the sequences and any varying CDR delineations obtained thereby.

PCSK-9 specific antagonists also have utility for various diagnostic purposes in the detection and quantification of PCSK9.

The following examples are provided to illustrate the present invention without limiting the same hereto:

### EXAMPLE 1

Characterization of the PCSK9 Antagonists

The PCSK9 antagonists used in this assay were antibodies E07, G08 and H23. G08 is disclosed in WO2008057459, which is incorporated in its entirety herein.

The sequences of E07 and H23 are as follows:

E07
IgG Vl3_3 light chain nucleotide sequence PCSK9_5_CX3_E07
(SEQ ID NO: 1)
(underlined residues are CDRs)
GATATCGAACTGACCCAGCCGCCTTCAGTGAGCGTTGCACCAGGTCAGACCGCGCG
TATCTCGTGTAGCGGCGATTCTCTTCGTGATAAGTATGTTCATTGGTACCAGCAG
AAACCCGGGCAGGCGCCAGTTGTTGTGATTTATTATGATACTAATCGTCCCTCAG
GCATCCCGGAACGCTTTAGCGGATCCAACAGCGGCAACACCGCGACCCTGACCATT
AGCGGCACTCAGGCGGAAGACGAAGCGGATTATTATTGCGCTGCTTATACTCGTTC
TATTTATGTGTTTGGCGGCGGCACGAAGTTAACCGTTCTTGGCCAGCCCAAGGCCA
ACCCCACCGTGACCCTGTTCCCCCCATCTTCTGAGGAGCTGCAAGCCAACAAGGCCA
CCCTGGTGTGCCTGATCTCTGACTTCTACCCTGGCGCTGTGACAGTGGCCTGGAAGG
CTGATGGCTCTCCTGTGAAGGCTGGCGTGGAGACCACCAAGCCATCTAAGCAGTCTA
ACAACAAGTATGCTGCCTCTTCTTACCTGTCTCTGACCCCTGAGCAGTGGAAGAGCC
ACCGGTCTTACTCTTGCCAGGTGACCCATGAGGGCTCTACAGTGGAGAAGACAGTG
GCCCCCACAGAGTGCTCT
IgG2M4 VH3_3 heavy chain nucleotide sequence PCSK9_5_CX3_E07
(SEQ ID NO: 2)
(underlined residues are CDRs)
CAGGTGCAATTGGTGGAAAGCGGCGGCGGCCTGGTGCAACCGGGCGGCAGCCTGCG
TCTGAGCTGCGCGGCCTCCGGATTTACCTTTTCTGATCATTGGATGCATTGGGTGC
GCCAAGCCCCTGGGAAGGGTCTCGAGTGGGTGAGCTATATCGATTATTATGGTAG
CAATACCCATTATGCGGATAGCGTGAAAGGCCGTTTTACCATTTCACGTGATAATT
CGAAAAACACCCTGTATCTGCAAATGAACAGCCTGCGTGCGGAAGATACGGCCGTG
TATTATTGCGCGCGTATGCTTTATGGTTGGAATTATGGTGTTTTTGATTATTGGGG
CCAAGGCACCCTGGTGACGGTTAGCTCAGCATCCACCAAGGGCCCATCCGTCTTCCC
CCTGGCGCCCTGCTCCAGGAGCACCTCCGAGAGCACAGCCGCCCTGGGCTGCCTGGT
CAAGGACTACTTCCCCGAACCGGTGACGGTGTCGTGGAACTCAGGCGCCCTGACCA
GCGGCGTGCACACCTTCCCGGCTGTCCTACAGTCCTCAGGACTCTACTCCCTCAGCA
GCGTGGTGACCGTGACCTCCAGCAACTTTGGCACGCAGACCTACACCTGCAACGTA
GATCACAAGCCCAGCAACACCAAGGTGGACAAGACAGTTGAGCGGAAATGCTGCGT
GGAGTGCCCACCATGCCCAGCACCTCCAGTGGCCGGACCATCAGTCTTCCTGTTCCC
CCCAAAACCCAAGGACACTCTCATGATCTCCCGGACCCCTGAGGTCACGTGCGTGGT
GGTGGACGTGAGCCAGGAAGACCCCGAGGTCCAGTTCAACTGGTACGTGGATGGCG
TGGAGGTGCATAATGCCAAGACAAAGCCGCGGGAGGAGCAGTTCAACAGCACGTTC
CGTGTGGTCAGCGTCCTCACCGTCCTGCACCAGGACTGGCTGAACGGCAAGGAGTA
CAAGTGCAAGGTCTCCAACAAAGGCCTCCCGTCCTCCATCGAGAAAACCATCTCCA
AAACCAAAGGGCAGCCCCGAGAGCCACAGGTGTACACCCTGCCCCCATCCCGGGAG
GAGATGACCAAGAACCAGGTCAGCCTGACCTGCCTGGTCAAAGGCTTCTACCCCAG
CGACATCGCCGTGGAGTGGGAGAGCAATGGGCAGCCGGAGAACAACTACAAGACC
ACGCCTCCCATGCTGGACTCCGACGGCTCCTTCTTCCTCTACAGCAAGCTAACCGTG
GACAAGAGCAGGTGGCAGCAGGGGAATGTCTTCTCATGCTCCGTGATGCATGAGGC
TCTGCACAACCACTACACACAGAAGAGCCTCTCCCTGTCTCCTGGTAAA
IgG Vl3_3 light chain amino acid sequence PCSK9_5_CX3_E07
(SEQ ID NO: 3)
(underlined residues are CDRs)
DIELTQPPSVSVAPGQTARISCSGDSLRDKYVHWYQQKPGQAPVVVIYYDTNRPSGIPE
RFSGSNSGNTATLTISGTQAEDEADYYCAAYTRSIYVFGGGTKLTVLGQPKANPTVTLFP
PSSEELQANKATLVCLISDFYPGAVTVAWKADGSPVKAGVETTKPSKQSNNKYAASSYL
SLTPEQWKSHRSYSCQVTHEGSTVEKTVAPTECS
IgG2m4 VH3_3 heavy chain amino acid sequence PCSK9_5_CX3_E07
(SEQ ID NO: 4)
(underlined residues are CDRs)
QVQLVESGGGLVQPGGSLRLSCAASGFTFSDHWMHWVRQAPGKGLEWVSYIDYYGS
NTHYADSVKGRFTISRDNSKNTLYLQMNSLRAEDTAVYYCARMLYGWNYGVFDYWG
QGTLVTVSSASTKGPSVFPLAPCSRSTSESTAALGCLVKDYFPEPVTVSWNSGALTSGVH
TFPAVLQSSGLYSLSSVVTVTSSNFGTQTYTCNVDHKPSNTKVDKTVERKCCVECPPCP
APPVAGPSVFLFPPKPKDTLMISRTPEVTCVVVDVSQEDPEVQPNWYVDGVEVHNAKTK
PREEQFNSTFRVVSVLTVLHQDWLNGKEYKCKVSNKGLPSSIEKTISKTKGQPREPQVYT
LPPSREEMTKNQVSLTCLVKGFYPSDIAVEWESNGQPENNYKTTPPMLDSDGSFFLYSKL
TVDKSRWQQGNVFSCSVMHEALHNHYTQKSLSLSPGK
H23
IgG Vk3_3b light chain nucleotide sequence PCSK9_6_CX1_H23
(SEQ ID NO: 5)
(underlined residues are CDRs)
GATATCGTGCTGACCCAGAGCCCGGCGACCCTGAGCCTGTCTCCGGGCGAACGTGC
GACCCTGAGCTGCAGAGCGAGCCAGTCTGTTAATTCTAATTATCTGGCTTGGTAC
CAGCAGAAACCAGGTCAAGCACCGCGTCTATTAATTTATGGTGCTTCTTCTCGTG
CAACTGGGGTCCCGGCGCGTTTTAGCGGCTCTGGATCCGGCACGGATTTTACCCTGA
CCATTAGCAGCCTGGAACCTGAAGACTTTGCGGTTTATTATTGCCAGCAGTGGGGT
GATGTTCCTATTACCTTTGGCCAGGGTACGAAAGTTGAAATTAAACGTACGGTGGC
TGCACCATCTGTCTTCATCTTCCCGCCATCTGATGAGCAGTTGAAATCTGGAACTGC
CTCTGTTGTGTGCCTGCTGAATAACTTCTATCCCAGAGAGGCCAAAGTACAGTGGAA
GGTGGATAACGCCCTCCAATCGGGTAACTCCCAGGAGAGTGTCACAGAGCAGGACA
GCAAGGACAGCACCTACAGCCTCAGCAGCACCCTGACGCTGAGCAAAGCAGACTAC
GAGAAACACAAAGTCTACGCCTGCGAAGTCACCCATCAGGGCCTGAGCTCGCCCGT
CACAAAGAGCTTCAACAGGGGAGAGTGT
IgG2M4 VH3_3 heavy chain nucleotide sequence PCSK9_6_CX1_H23
(SEQ ID NO: 6)
(underlined residues are CDRs)
CAGGTGCAATTGGTGGAAAGCGGCGGCGGCCTGGTGCAACCGGGCGGCAGCCTGCG
TCTGAGCTGCGCGGCCTCCGGATTTACCTTTTCTGATTATTATATGCATTGGGTGC
GCCAAGCCCCTGGGAAGGGTCTCGAGTGGGTGAGCAATATCTCTGGTTCTGGTAG
CACTACCTATTATGCGGATAGCGTGAAAGGCCGTTTTACCATTTCACGTGATAATT
CGAAAAACACCCTGTATCTGCAAATGAACAGCCTGCGTGCGGAAGATACGGCCGTG
TATTATTGCGCGCGTGGTATGTTTGATTTTTGGGGCCAAGGCACCCTGGTGACGGT
TAGCTCAGCATCCACCAAGGGCCCATCCGTCTTCCCCCTGGCGCCCTGCTCCAGGAG
CACCTCCGAGAGCACAGCCGCCCTGGGCTGCCTGGTCAAGGACTACTTCCCCGAACC
GGTGACGGTGTCGTGGAACTCAGGCGCCCTGACCAGCGGCGTGCACACCTTCCCGG
CTGTCCTACAGTCCTCAGGACTCTACTCCCTCAGCAGCGTGGTGACCGTGACCTCCA
GCAACTTTGGCACGCAGACCTACACCTGCAACGTAGATCACAAGCCCAGCAACACC
AAGGTGGACAAGACAGTTGAGCGGAAATGCTGCGTGGAGTGCCCACCATGCCCAGC
ACCTCCAGTGGCCGGACCATCAGTCTTCCTGTTCCCCCCAAAACCCAAGGACACTCT
CATGATCTCCCGGACCCCTGAGGTCACGTGCGTGGTGGTGGACGTGAGCCAGGAAG
ACCCCGAGGTCCAGTTCAACTGGTACGTGGATGGCGTGGAGGTGCATAATGCCAAG
ACAAAGCCGCGGGAGGAGCAGTTCAACAGCACGTTCCGTGTGGTCAGCGTCCTCAC
CGTCCTGCACCAGGACTGGCTGAACGGCAAGGAGTACAAGTGCAAGGTCTCCAACA
AAGGCCTCCCGTCCTCCATCGAGAAAACCATCTCCAAAACCAAAGGGCAGCCCCGA
GAGCCACAGGTGTACACCCTGCCCCCATCCCGGGAGGAGATGACCAAGAACCAGGT
CAGCCTGACCTGCCTGGTCAAAGGCTTCTACCCCAGCGACATCGCCGTGGAGTGGG
AGAGCAATGGGCAGCCGGAGAACAACTACAAGACCACGCCTCCCATGCTGGACTCC
GACGGCTCCTTCTTCCTCTACAGCAAGCTAACCGTGGACAAGAGCAGGTGGCAGCA
GGGGAATGTCTTCTCATGCTCCGTGATGCATGAGGCTCTGCACAACCACTACACACA
GAAGAGCCTCTCCCTGTCTCCTGGTAAA
IgG Vk3_3b light chain amino acid sequence PCSK9_6_CX1_H23
(SEQ ID NO: 7)
(underlined residues are CDRs)
DIVLTQSPATLSLSPGERATLSCRASQSVNSNYLAWYQQKPGQAPRLLIYGASSRATGV
PARFSGSGSGTDFTLTISSLEPEDFAVYYCQQWGDYPITFGQGTKVEIKRTVAAPSVFIFP
PSDEQLKSGTASVVCLLNNFYPREAKVQWKVDNALQSGNSQESVTEQDSKDSTYSLSST
LTLSKADYEKHKVYACEVTHQGLSSPVTKSFNRGEC
IgG2m4 VH3_3 heavy chain amino acid sequence PCSK9_6_CX1_H23
(SEQ ID NO: 8)
(underlined residues are CDRs)
QVQLVESGGGLVQPGGSLRLSCAASGFTFSDYYMHWVRQAPGKGLEWVSNISGSGST
TYYADSVKGRFTISRDNSKNTLYLQMNSLRAEDTAVYYCARGMFDFWGQGTLVTVSS
ASTKGPSVFPLAPCSRSTSESTAALGCLVKDYFPEPVTVSWNSGALTSGVHTFPAVLQSS
GLYSLSSVVTVTSSNFGTQTYTCNVDHKPSNTKVDKTVERKCCVECPPCPAPPVAGPSV
FLFPPKPKDTLMISRTPEVTCVVVDVSQEDPEVQFNWYVDGVEVHNAKTKPREEQFNST
FRVVSVLTVLHQDWLNGKEYKCKVSNKGLPSSIEKTISKTKGQPREPQVYTLPPSREEMT
KNQVSLTCLVKGFYPSDIAVEWESNGQPENNYKTTPPMLDSDGSFFLYSKLTVDKSRWQ
QGNVFSCSVMHEALHNHYTQKSLSLSPGK

### EXAMPLE 2

Production of the 3E07 and 1 H23 Antibodies

DNA encoding the heavy chain variable regions were fused in-frame with DNA encoding the IgG2M4 constant region whereas DNA encoding the light chain variable regions were fused in-frame with DNA encoding either lambda or kappa light chain constant region in alignment with the corresponding variable regions. The cloning procedure is described below. The light chain vector comprises cloning sites flanked by a human CMV (HCMV) promoter and leader sequence on the 5′ end of one cloning site and the light chain constant region sequences and bovine growth hormone (BGH) pA polyadenylation signal on the 3′ side of the other cloning site. The heavy chain IgG2M4 constant region vector comprises cloning sites flanked by an HCMV promoter and leader sequence on the 5′ end of one cloning site and heavy chain IgG2M4 sequences and BGH pA polyadenylation signal on the 3′ side of the other cloning site. The expression vectors carry oriP from Epstein Barr virus (EBV) viral genome for prolonged expression in 293EBNA cells and the bacterial sequences for kanamycin selection marker and replication origin in E. coli. The leader sequence at the amino termini of the antibodies mediated the secretion of the expressed antibodies into the culture medium. The leader sequence for heavy chain is MEWSWVFLFFLSVTTGVHS (SEQ ID NO: 9) and light chain: MSVPTQVLGLLLLWLTDARC (SEQ ID NO:10).

The respective variable regions were PCR amplified in a volume of 25 containing high fidelity PCR master mix, template volume 1 μL and forward and reverse primers: 1μL each. PCR conditions were one cycle of 94° C. for two minutes, 25 cycles of 94° C. for 1.5 minutes, 60° C. for 1.5 minutes and 72° C. for 1.5 minutes with a final extension at 72° C. for 7 minutes. The amplified light and heavy chain variable region PCR products were cloned in-frame with the appropriate leader sequence at the 5′-end and constant region at the 3′-end using In-Fusion strategy (Clontech, Palo Alto, Calif.) and cloned into E. coil XL10 cells from Stratagene, La Jolla, Calif.). The DNA sequences for the clones were confirmed by sequencing and the amino acid sequences were deduced from the DNA sequences.

The above plasmids were transfected into 293EBNA monolayer cells using FUGENE transfection reagents (FUGENE is a trademark of Fugent LLC and is available from Roche Diagnostics, Nutley, N.J.). The transfected cells were incubated in OPTI-MEM serum free medium (Invitrogen) and the secreted antibodies were purified from the culture medium using protein A/G affinity chromatography. The concentration of purified antibodies was determined by OD at 280 nm and the purity by LABCHIP capillary SDS gel electrophoresis (Caliper Life Sciences, Hopkinton, Mass.). The antibodies purified were used for characterization described elsewhere.

EXAMPLE 3

Functional Analysis of E07, G08 and H23 Fab

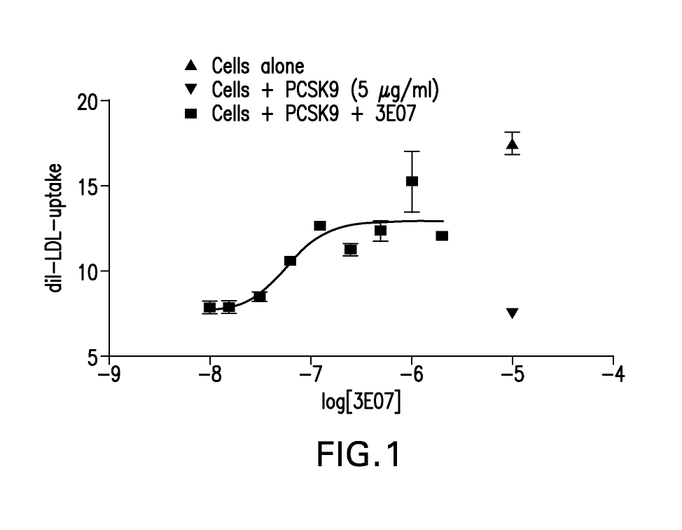

30,000 HEK293 cells/well were seeded in normal serum conditions and 24 hours later, media was changed to one lacking serum. 24 hours after that, LDL uptake was measured. 3E07 Fab was titrated with 5 ug/ml of hPCSK9 purified protein, starting at 100 ug/ml. The data in FIG. 1 demonstrate that E07 Fab is a partial inhibitor of PCSK9 function. The E07 Fab displays about 50% inhibition on the effect of hPCSK9.

EXAMPLE 4

E07, H23 and G08 Do Not Compete with Binding of a Known PCSK9 Antagonist (“1B20”)

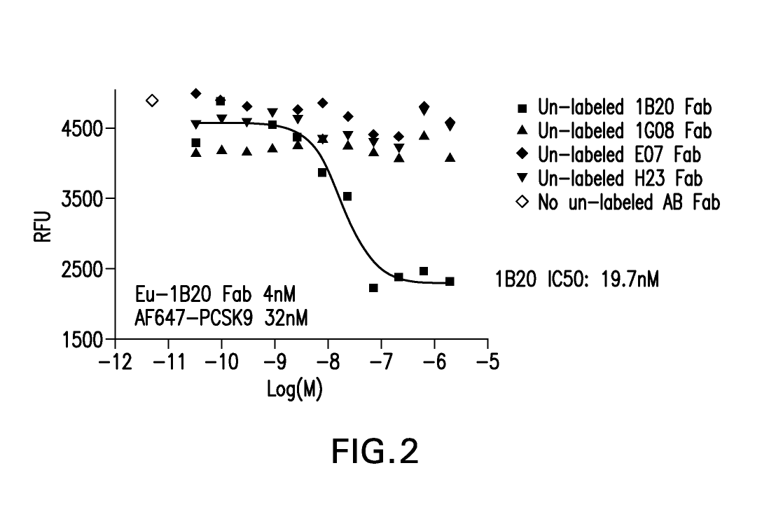

As seen in FIGS. 2, E07, G08 and H23 (Fab) do not compete with 1B20 IgG for

PCSK9 binding. The 1B20 antagonist is covered in U.S. Provisional Application No. 61/063,980 and related applications, and incorporated in its entirety herein. For this experiment, 4 nM (final concentration) of Eu-1G08 Fab was mixed with 32 nM (final concentration) of AF647-PCSK9 and various concentrations (from 1 μM to 50 pM) of unlabeled 1B20, G08, E07 and H23 Fab in 50 μl of assay buffer (10 mM HEPES pH 7.4, 150 mM NaCl, 0.05% BSA, 100 μM CaCl₂) in a black U-Bottom shaped pigmented styrene 96-well microtiter plate (Dynatech). The mixtures were incubated at room temperature for 3 hours and plate was read on a Ruby Star fluorescent reader (available from BMG Technologies, Inc.) at Ex 370 mm. Signals were recorded at both 620 mm and 665 mm. The 665 mm/620 mm ratio was used to calculate the results. The experiments were performed in triplicate and repeated 3 times. The background of the assay is ˜2340 RFU.

What is claimed is:

1. An isolated PCSK9-specific antagonist that antagonizes PCSK9's inhibition of cellular LDL uptake comprising a monoclonal antibody comprising a light chain polypeptide having the amino acid sequence of SEQ ID NO:3 and a heavy chain polypeptide having the amino acid sequence of SEQ ID NO: 4.

2. An isolated PCSK9-specific antagonist that antagonizes PCSK9's inhibition of cellular LDL uptake comprising a monoclonal antibody comprising a light chain polypeptide having the amino acid sequence of SEQ ID NO: 7 and a heavy chain polypeptide having the amino acid sequence of SEQ ID NO: 8.

3. The antagonist of PCSK-9 function of claim 1 which comprises a light chain polypeptide comprising CDR1, CDR2 and CDR3 of SEQ ID NO: 3 and a heavy chain polypeptide comprising CDR1, CDR2, and CDR3 of SEQ ID NO: 4.

4. The antagonist of PCSK-9 function of claim 1 which comprises a light chain polypeptide comprising CDR1, CDR2 and CDR3 of SEQ ID NO: 7 and a heavy chain polypeptide comprising CDR1, CDR2, and CDR3 of SEQ ID NO: 8.

5. A composition comprising the antagonist of PCSK-9 function of claim 3.

6. A composition comprising the antagonist of PCSK-9 function of claim 4.

7. A method for antagonizing PCSK9 function which comprises the step of administering a PCSK9-specific antagonist of claim 1.

8. A method for antagonizing PCSK9 function which comprises the step of administering a PCSK9-specific antagonist of claim 2.

9. Isolated nucleic acids coding for a monoclonal antibody comprising a light chain polypeptide having the nucleotide sequence of SEQ ID NO: 1 and a heavy chain polypeptide having the amino acid sequence of SEQ ID NO: 2.

10. Isolated nucleic acids coding for a monoclonal antibody comprising a light chain polypeptide having the nucleotide sequence of SEQ ID NO: 5 and a heavy chain polypeptide having the amino acid sequence of SEQ ID NO: 6.

11. A vector comprising isolated nucleic acid coding for the polypeptide of claim 1.

12. A vector comprising isolated nucleic acid coding for the polypeptide of claim 2.

13. A host cell comprising the vector of claim 11.

14. A host cell comprising the vector of claim 12.

15. A method for producing a PCSK9-specific antagonist which comprises:
(a) culturing a population of cells comprising the cell of claim 13 under conditions appropriate for production of the PCSK9-specific antagonist; and
(b) isolating the PCSK9-specific antagonist produced.

16. A method for producing a PCSK9-specific antagonist which comprises:
(a) culturing a population of cells comprising the cell of claim 14 under conditions appropriate for production of the PCSK9-specific antagonist; and
(b) isolating the PCSK9-specific antagonist produced.


SEQUENCE LISTING (10 sequences)

SEQ ID NO: 1 - DNA, 636 bp, Artificial Sequence
  LC; 5CX3E07
gatatcgaactgacccagccgccttcagtgagcgttgcaccaggtcagaccgcgcgtatctcgtgtagcggcgattctcttcgtgataagtatgttcattggtaccagcagaaacccgggcaggcgccagttgttgtgatttattatgatactaatcgtccctcaggcatcccggaacgctttagcggatccaacagcggcaacaccgcgaccctgaccattagcggcactcaggcggaagacgaagcggattattattgcgctgcttatactcgttctatttatgtgtttggcggcggcacgaagttaaccgttcttggccagcccaaggccaaccccaccgtgaccctgttccccccatcttctgaggagctgcaagccaacaaggccaccctggtgtgcctgatctctgacttctaccctggcgctgtgacagtggcctggaaggctgatggctctcctgtgaaggctggcgtggagaccaccaagccatctaagcagtctaacaacaagtatgctgcctcttcttacctgtctctgacccctgagcagtggaagagccaccggtcttactcttgccaggtgacccatgagggctctacagtggagaagacagtggcccccacagagtgctct

SEQ ID NO: 2 - DNA, 1341 bp, Artificial Sequence
  VH; 5CX3E07
caggtgcaattggtggaaagcggcggcggcctggtgcaaccgggcggcagcctgcgtctgagctgcgcggcctccggatttaccttttctgatcattggatgcattgggtgcgccaagcccctgggaagggtctcgagtgggtgagctatatcgattattatggtagcaatacccattatgcggatagcgtgaaaggccgttttaccatttcacgtgataattcgaaaaacaccctgtatctgcaaatgaacagcctgcgtgcggaagatacggccgtgtattattgcgcgcgtatgctttatggttggaattatggtgtttttgattattggggccaaggcaccctggtgacggttagctcagcatccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgacctccagcaactttggcacgcagacctacacctgcaacgtagatcacaagcccagcaacaccaaggtggacaagacagttgagcggaaatgctgcgtggagtgcccaccatgcccagcacctccagtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaaggcctcccgtcctccatcgagaaaaccatctccaaaaccaaagggcagccccgagagccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctaaccgtggacaagagcaggtggcagcaggggaatgtcttctcatgctccgtgatgcatgaggctctgcacaaccactacacacagaagagcctctccctgtctcctggtaaa

SEQ ID NO: 3 - protein, 212 aa, Artificial Sequence
  LC; 5CX3E07
DIELTQPPSVSVAPGQTARISCSGDSLRDKYVHWYQQKPGQAPVVVIYYDTNRPSGIPERFSGSNSGNTATLTISGTQAEDEADYYCAAYTRSIYVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 4 - protein, 447 aa, Artificial Sequence
  VH; 5CX3E07
QVQLVESGGGLVQPGGSLRLSCAASGFTFSDHWMHWVRQAPGKGLEWVSYIDYYGSNTHYADSVKGRFTISRDNSKNTLYLQMNSLRAEDTAVYYCARMLYGWNYGVFDYWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - DNA, 645 bp, Artificial Sequence
  LC; 6CX1H23
gatatcgtgctgacccagagcccggcgaccctgagcctgtctccgggcgaacgtgcgaccctgagctgcagagcgagccagtctgttaattctaattatctggcttggtaccagcagaaaccaggtcaagcaccgcgtctattaatttatggtgcttcttctcgtgcaactggggtcccggcgcgttttagcggctctggatccggcacggattttaccctgaccattagcagcctggaacctgaagactttgcggtttattattgccagcagtggggtgatgttcctattacctttggccagggtacgaaagttgaaattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 6 - DNA, 1320 bp, Artificial Sequence
  VH; 6CX1H23
caggtgcaattggtggaaagcggcggcggcctggtgcaaccgggcggcagcctgcgtctgagctgcgcggcctccggatttaccttttctgattattatatgcattgggtgcgccaagcccctgggaagggtctcgagtgggtgagcaatatctctggttctggtagcactacctattatgcggatagcgtgaaaggccgttttaccatttcacgtgataattcgaaaaacaccctgtatctgcaaatgaacagcctgcgtgcggaagatacggccgtgtattattgcgcgcgtggtatgtttgatttttggggccaaggcaccctggtgacggttagctcagcatccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgacctccagcaactttggcacgcagacctacacctgcaacgtagatcacaagcccagcaacaccaaggtggacaagacagttgagcggaaatgctgcgtggagtgcccaccatgcccagcacctccagtggccggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgttccgtgtggtcagcgtcctcaccgtcctgcaccaggactggctgaacggcaaggagtacaagtgcaaggtctccaacaaaggcctcccgtcctccatcgagaaaaccatctccaaaaccaaagggcagccccgagagccacaggtgtacaccctgcccccatcccgggaggagatgaccaagaaccaggtcagcctgacctgcctggtcaaaggcttctaccccagcgacatcgccgtggagtgggagagcaatgggcagccggagaacaactacaagaccacgcctcccatgctggactccgacggctccttcttcctctacagcaagctaaccgtggacaagagcaggtggcagcaggggaatgtcttctcatgctccgtgatgcatgaggctctgcacaaccactacacacagaagagcctctccctgtctcctggtaaa

SEQ ID NO: 7 - protein, 215 aa, Artificial Sequence
  LC; 6CX1H23
DIVLTQSPATLSLSPGERATLSCRASQSVNSNYLAWYQQKPGQAPRLLIYGASSRATGVPARFSGSGSGTDFTLTISSLEPEDFAVYYCQQWGDVPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 440 aa, Artificial Sequence
  VH; 6CX1H23
QVQLVESGGGLVQPGGSLRLSCAASGFTFSDYYMHWVRQAPGKGLEWVSNISGSGSTTYYADSVKGRFTISRDNSKNTLYLQMNSLRAEDTAVYYCARGMFDFWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 19 aa, Artificial Sequence
  leader sequence for heavy chain
MEWSWVFLFFLSVTTGVHS

SEQ ID NO: 10 - protein, 20 aa, Artificial Sequence
  leader sequence for light chain
MSVPTQVLGLLLLWLTDARC